(19) United States
(12) Patent Application Publication
(10) Pub. No.: US 2023/0069554 A1
(43) Pub. Date: Mar. 2, 2023

(54) ASSAY METHOD FOR ANTIMICROBIAL ACTIVITY OF ANTIMICROBIAL AGENT

(71) Applicant: LG ELECTRONICS INC., Seoul (KR)

(72) Inventors: Jegoo SON, Seoul (KR); Sunyoung PARK, Seoul (KR)

(21) Appl. No.: 17/797,431

(22) PCT Filed: Dec. 10, 2020
(86) PCT No.: PCT/KR2020/018098

§ 371 Date: Aug. 4, 2022

(30) Foreign Application Priority Data
Feb. 4, 2020 (KR) 10-2020-0013182

(51) Int. Cl. C12Q 1/18; A01N 25/12; A01N 59/00; C12N 1/20

(52) U.S. Cl. CPC C12Q 1/18; A01N 25/12; A01N 59/00; C12N 1/20

(57) ABSTRACT

The present disclosure relates to an assay method for the antimicrobial activity of an antimicrobial agent. The assay method for the antimicrobial activity of an antimicrobial agent according to the present disclosure comprises: injecting an antimicrobial agent into a culture medium in which a strain is grown; shaking the culture medium according to time and sampling the culture medium according to the time; after the sampling, centrifuging the culture medium into which the antimicrobial agent is injected; and after the centrifuging, measuring the number of microorganisms after culturing a bacterial solution from which the antimicrobial agent is removed, wherein the antimicrobial activity can be evaluated depending on the type and content of the antimicrobial agent, and the antimicrobial activity of the antimicrobial agent can be accurately and reliably evaluated.

### TECHNICAL FIELD

Disclosed herein is an assay method for the antimicrobial activity of an antimicrobial agent, enabling the assay of antimicrobial activity depending on the sort and content of an antimicrobial agent and the assay of the antimicrobial activity of an antimicrobial agent accurately and reliably.

### BACKGROUND ART

In recent years, antimicrobial processing has been applied to a wide range of industrial products, and the antimicrobial processing industry expands rapidly. At the advent of antimicrobial processing, antimicrobial processing targeted medical devices. Currently, however, antimicrobial processing is applied to a variety of products ranging from home appliances, stationeries, furniture to steel products.

Previously, a synthetic organic antimicrobial agent or an Ag ion-based inorganic antimicrobial agent was usually used. Later, various types of antimicrobial agents have been used at a time when issues such as human friendliness, an environmentally friendly processing method, and the minimization of a human health risk and the like attracts public attention.

It turned out that conventional synthetic organic antimicrobial agents and Ag ion-based inorganic antimicrobial agents show a relative high anti microbial activity. However, the antimicrobial activity of an antimicrobial agent itself varies depending on testers. Additionally, even minimum inhibitory concentrations (MIC) against bacteria are not determined accurately.

The reason the MIC of an antimicrobial agent is not determined as a constant value is that there is no assay method for antimicrobial activity unique to an antimicrobial agent, and if it is doubtful whether an antimicrobial agent itself shows antimicrobial activity, a product, processed with the antimicrobial agent, lacks reliability in its antimicrobial activity.

For this reason, there is a growing demand for a test method for accurately measuring the antimicrobial activity of an antimicrobial agent itself.

An evaluation method of quantitative antimicrobial efficiency of an antimicrobial sample based on the disc diffusion method, and a measuring apparatus are disposed in KR Patent No. 10-1439918 (published on Sep. 15, 2014). However, the disclosure presents a method of evaluating the antimicrobial activity of an antimicrobial agent quantitatively, rather than a method of evaluating the antimicrobial activity of an antimicrobial agent itself accurately and objectively.

### DESCRIPTION OF INVENTION

### Technical Problems

The objective of the present disclosure is to provide an assay method for the antimicrobial activity of an antimicrobial agent, by which time taken for an antimicrobial agent to exhibit antimicrobial activity can be measured, and an excellent antimicrobial agent among antimicrobial agents can be selected.

Aspects according to the present disclosure are not limited to the above ones, and other aspects and advantages that are not mentioned above can be clearly understood from the following description and can be more clearly understood from the embodiments set forth herein. Additionally, the aspects and advantages in the present disclosure can be realized via means and combinations thereof that are described in the appended claims.

### Technical Solutions

In the present disclosure, an assay method for the antimicrobial activity of an antimicrobial agent includes sampling an antimicrobial agent into which strains are injected, temporally, at a time of shaking, such that the number of viable microorganisms are repeatedly checked temporally, thereby understanding the tendency of the antimicrobial activity of the antimicrobial agent.

The assay method for the antimicrobial activity of an antimicrobial agent includes a centrifuging process in which the antimicrobial agent separates from a bacterial solution after the sampling, to prevent the strains from being exposed to the antimicrobial agent continuously, thereby improving accuracy in the antimicrobial activity of the antimicrobial agent.

Specifically, the assay method for the antimicrobial activity of an antimicrobial agent includes injecting an antimicrobial agent into a culture medium where strains are grown; shaking the culture medium according to time and sampling the culture medium according to the time; after the sampling, centrifuging the culture medium into which the antimicrobial agent is injected; and after the centrifuging, culturing a bacterial solution from which the antimicrobial agent is removed and then measuring the number of microorganisms.

At this time, the antimicrobial agent is applied to powder as well as fabric, and although a small amount of the antimicrobial agent is applied to the culture medium where the strains are grown, the antimicrobial activity of the antimicrobial agent can be evaluated accurately and objectively.

### Advantageous Effects

According to the present disclosure, shaking and sampling are performed at specific time intervals, and the tendency of the antimicrobial activity of an antimicrobial agent is objectively found, making it possible to accurately measure time taken for the antimicrobial agent to exhibit antimicrobial activity.

Additionally, a small amount of an antimicrobial agent is used to accurately determine the antimicrobial activity of the antimicrobial agent, and it can be determined whether an antimicrobial agent shows antimicrobial activity or not, and a small amount of an antimicrobial agent can be used to sort its antimicrobial activity.

Further, a process of separating an antimicrobial agent from a bacterial solution is included, making it possible to improve accuracy in antimicrobial activity, and various types of powdered antimicrobial agents can be applied to various materials without being limited to a material such as fabrics and can be evaluated.

### BRIEF DESCRIPTION OF DRAWINGS

FIG. 1 is a flow chart showing an assay method for the antimicrobial activity of an antimicrobial agent according to the present disclosure.

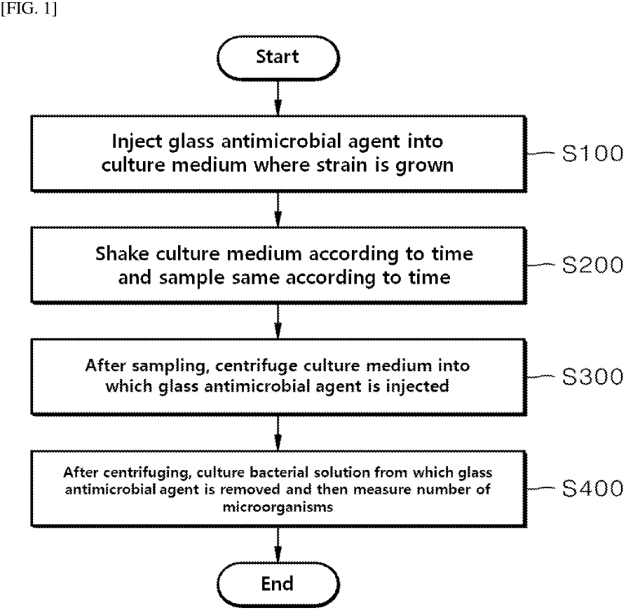

FIG. 2 is a graph showing antimicrobial activity of a sample, based on a conventional shake culture method.

FIG. 3 is a graph showing antimicrobial activity of a sample, based on the assay method of antimicrobial activity of an antimicrobial agent according to the present disclosure.

### DETAILED DESCRIPTION OF EXEMPLARY EMBODIMENTS

The above-described aspects, features and advantages are specifically described hereafter such that one having ordinary skill in the art can embody the technical spirit of the disclosure easily. In the disclosure, detailed description of known technologies in relation to the disclosure is omitted if it is deemed to make the gist of the disclosure unnecessarily vague. Below, preferred embodiments according to the disclosure are specifically described.

Embodiments are not limited to the embodiments set forth herein, and can be modified and changed in various different forms. The embodiments in the disclosure are provided such that the disclosure can be through and complete and the scope of the disclosure can be fully conveyed to one of ordinary skill in the art.

When one component is described as being “in the upper portion (or the lower portion)” or “on (or under)” another component, one component can be directly on (or under) another component, and an additional component can be interposed between the two components.

When any one component is described as being “connected”, “coupled”, or “connected” to another component, any one component can be directly connected or coupled to another component, but an additional component can be “interposed” between the two components or the two components can be “connected”, “coupled”, or “connected” by an additional component.

In the disclosure, singular forms include plural forms as well, unless explicitly indicated otherwise. It is to be understood that the terms such as “comprise” or “include” and the like, when used in this disclosure, are not interpreted as necessarily including stated components or steps, but can be interpreted as excluding some of the stated components or steps or as further including additional components or steps.

In the disclosure, singular forms include plural forms as well, unless explicitly indicated otherwise. It is to be understood that the terms such as “comprise” or “include” and the like, when used in this disclosure, are not interpreted as necessarily including stated components or steps, but can be interpreted as excluding some of the stated components or steps or as further including additional components or steps.

Throughout the disclosure, the terms “A and/or B” as used herein can denote A, B or A and B, and the terms “C to D” can denote C or greater and D or less, unless stated to the contrary.

Hereafter, an assay method for the antimicrobial activity of an antimicrobial agent according to the present disclosure is described specifically.

In the disclosure, provided is an assay method for the antimicrobial activity of an antimicrobial agent, comprising: injecting an antimicrobial agent into a culture medium in which a strain is grown;
shaking the culture medium temporally and sampling the culture medium temporally;
after the sampling, centrifuging the culture medium into which the antimicrobial agent is injected; and
after the centrifuging, culturing a bacterial solution from which the antimicrobial agent is removed and then measuring the number of microorganisms.

In the existing test for the antimicrobial activity of an antimicrobial agent (ASTM-E2149), the number of microorganisms in the initial stage and the number of microorganisms in the final stage are only quantified. Accordingly, it is difficult to accurately measure time taken for powders to exhibit antimicrobial activity. Additionally, since a relatively large amount of powder is used in the test for the antimicrobial activity of an antimicrobial agent, powders showing a different type of antimicrobial activity cannot be compared.

The assay method for the antimicrobial activity of an antimicrobial agent according to the present disclosure involves shaking an antimicrobial agent in a culture medium where a strain is grown temporally and sampling the culture medium temporally, thereby making it possible to accurately measure time taken for antimicrobial powders to exhibit antimicrobial activity, unlike existing methods.

Further, a relatively small amount of an antimicrobial agent is used to determine the antimicrobial activity of the antimicrobial agent accurately. Furthermore, it can be ascertained whether an antimicrobial agent exhibits antimicrobial activity, and a small amount of an antimicrobial agent can be used to sort its antimicrobial activity.

The assay method for the anti microbial activity of an antimicrobial agent according to the present disclosure includes injecting an antimicrobial agent into a culture medium where a strain is grown.

At this time, the antimicrobial activity of a powdered antimicrobial agent can be assayed in addition to the antimicrobial activity of an antimicrobial agent injected into a specific material such as a fabric sample.

In the assay method for the antimicrobial activity of an antimicrobial agent according to the present disclosure, 0.02 to 2 g of the antimicrobial agent may be injected into the culture medium where the strain is grown.

Regarding this, in the existing shake culture method (ASTM-E2149), 2 g of an antimicrobial agent is used for a test, and such an amount helps the antimicrobial agent to exhibit high antimicrobial activity.

Accordingly, in the existing shake culture method, it is difficult to determine the antimicrobial activity of an antimicrobial gent accurately. However, in the assay method for the antimicrobial activity of an antimicrobial agent according to the present disclosure, an antimicrobial agent is used in the above-described range of numbers. Thus, the antimicrobial performance of the antimicrobial agent itself can be evaluated objectively, and the antimicrobial performance of antimicrobial agents can be compared.

In the assay method for the antimicrobial activity of an antimicrobial agent according to the present disclosure, the strain may be a strain in electronic products, in particular, washing machines, air purifiers and the like, and specifically, may be one selected from a group consisting of Klebsiella pneumoniae, Staphylococcus aureus, Micrococcus, Slalmonella typhimurium, Escherichia coli, Bacillus subtillus and Pseudomonas aeruginosa.

The assay method for the antimicrobial activity of an antimicrobial agent according to the present disclosure includes shaking the culture medium temporally and sampling the culture medium temporally.

In the assay method for the antimicrobial activity of an antimicrobial agent according to the present disclosure, the culture medium is shaken temporally and sampling the culture medium is performed temporally, such that a change in the number of microorganisms is schematized temporally. Thus, the tendency of the antimicrobial activity of the antimicrobial agent can be found.

Sampling of the shaken culture medium may be performed at 20-minute intervals, and such temporal sampling may vary depending on the sort of an antimicrobial agent.

The assay method for the antimicrobial activity of an antimicrobial agent according to the present disclosure includes centrifuging the culture medium into which the antimicrobial agent is injected, after the sampling.

In the present disclosures, when the strain is kept exposed to the antimicrobial agent, the inaccuracy in results may improve, based on the centrifuging process, ensuring an accurate assay of antimicrobial activity.

The process of centrifuging is performed at 4000 to 6000 rpm, for 5 to 10 seconds, for example. The process of centrifuging at less than 4000 rpm results in no separation of the antimicrobial agent from a bacterial solution, and the process of centrifuging at greater than 6000 rpm results in consumption of excessive energy in the separation of the antimicrobial agent from a bacterial solution, causing deterioration in processing efficiency.

In the assay method for the antimicrobial activity of an antimicrobial agent according to the present disclosure, a powdered antimicrobial agent can be used, and centrifuging is possible. Thus, various types of antimicrobial agents can be applied to tests for antimicrobial activity.

The assay method for the antimicrobial activity of an antimicrobial agent according to the present disclosure includes culturing a bacterial solution from which the antimicrobial gent is removed and then measuring the number of microorganisms, after the centrifuging.

After the above-described centrifuging process, a separated bacterial solution is cultured to check the number of microorganisms with the naked eye, and the number of living microorganisms is measured to assay antimicrobial activity.

### Embodiment 1: Assay of Antimicrobial Activity of Antimicrobial Agent

### 1) Culture of Strain for Experiment

Strains of Klebsiella pneumonia were put in 50 ml of a sterilized LB culture solution, stirred in an incubator of 37° C. and cultured for 18 hours.

### 2) Injection of Strains

A powdered glass antimicrobial agent was put into a conical flask of 250 ml, and 1.5 to 3.0x105 CFU/ml of the cultured strains of Klebsiella pneumonia were injected into 50 ml of the sterilized LB culture solution.

### 3) Exposure to Shaking and Sampling

The glass antimicrobial agent into which the strains were injected was put into a shaker with arms (a wrist-action shaker), exposed to shaking at 37° C., for 20, 40 and 60 minutes and then sampled respectively.

### 4) Centrifuging

After the sampling, centrifuging was performed at 6000 rpm for 10 seconds, to return a bacterial solution that is a supernatant, and the bacterial solution was separated from the antimicrobial agent.

### 5) Culture

The separated bacterial solution was cultured on a standard agar plate medium for 24 to 48 hours, and the number of colonies, formed on the medium the day after the culture, was measured with a counting machine.

### Experimental Example 1: Assay of Antimicrobial Activity Based on Antimicrobial Gent

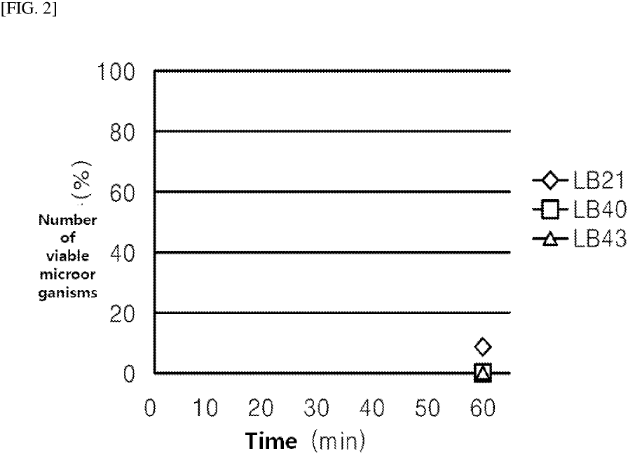

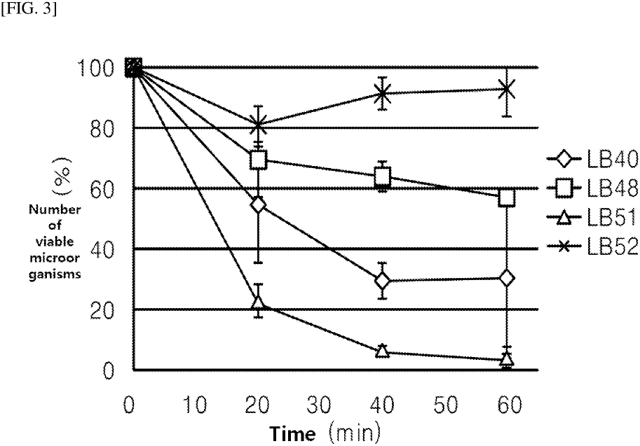

To see the precision and accuracy in the assay method for the antimicrobial activity of an antimicrobial gent according to the present disclosure, the antimicrobial activity of various samples were analyzed, using the existing shake culture method, and the assay method for the antimicrobial activity of an antimicrobial agent in the present disclosure, and results of the analysis are shown in FIGS. 2 and 3.

FIG. 2 is a graph showing the antimicrobial activity of samples A (a Zn-based antimicrobial agent), B (a Zn-based antimicrobial agent) and C (an Ag-based antimicrobial agent), based on the existing shake culture method (Samples A and B are both Zn-based antimicrobial agents but have a different Zn content.).

As shown in FIG. 2, a decrease in the number of viable microorganisms after an hour of shaking is only found according to the existing shake culture method.

FIG. 3 is a graph showing the antimicrobial activity of samples B (a Zn-based antimicrobial agent), D (an Ag-based antimicrobial agent), E (a Cu-based antimicrobial agent) and F (a Cu-based antimicrobial agent), based on the assay method for the antimicrobial activity of an antimicrobial agent in the present disclosure (Samples C and D are both Ag-based antimicrobial agents but have a different Ag content, and samples E and F are both Cu-based antimicrobial agents but has a different Cu content.).

Referring to FIG. 3, the assay method for the antimicrobial activity of an antimicrobial agent according to the present disclosure makes it possible to know the antimicrobial activity of an antimicrobial agent temporally, and measure antimicrobial activity depending on samples.

Thus, the assay method for the antimicrobial activity of an antimicrobial agent according to the present disclosure may help to accurately measure time taken for a powdered antimicrobial agent to exhibit antimicrobial activity, unlike the existing method.

Further, in the assay method for the antimicrobial activity of an antimicrobial agent according to the present disclosure, a small amount of an antimicrobial agent is used to determine the antimicrobial activity of the antimicrobial agent more accurately than in the existing method. Furthermore, it can be determined whether an antimicrobial agent shows antimicrobial activity or not, and a small amount of an antimicrobial agent can be used to sort its antimicrobial activity.

The embodiments are described above with reference to a number of illustrative embodiments thereof. However, embodiments are not limited to the embodiments and drawings set forth herein, and numerous other modifications and embodiments can be drawn by one skilled in the art within the technical scope of the disclosure. Further, the effects and predictable effects based on the configurations in the disclosure are to be included within the range of the disclosure though not explicitly described in the description of the embodiment.

1. An assay method for antimicrobial activity of an antimicrobial agent, comprising:
injecting the antimicrobial agent into a culture medium in which strains are grown;
shaking the culture medium according to time, and sampling the culture medium according to the time;
after the sampling of the culture medium, centrifuging the culture medium such that the antimicrobial agent is separated from a bacterial solution; and
after the centrifuging of the culture medium, culturing theft bacterial solution in separated from the antimicrobial agent and then determining a number of microorganisms.

2. The assay method of claim 1, wherein the antimicrobial agent is a powdered antimicrobial agent.

3. The assay method of claim 1, wherein the injecting of the antimicrobial agent includes injecting 0.02 g to 2 g of the antimicrobial agent into the culture medium.

4. The assay method of claim 1, wherein the strains are one selected from a group consisting of Klebsiella pneumoniae, Staphylococcus aureus, Micrococcus, Slalmonella typhimurium, Escherichia coli, Bacillus subtillus and Pseudomonas aeruginosa.

5. The assay method of claim 1, wherein the sampling of the culture medium includes sampling of the culture medium according to time such that a change in the number of microorganisms is schematized temporally.

6. The assay method of claim 1, wherein the sampling is performed at 20-minute intervals.

7. The assay method of claim 1, wherein the culture medium is centrifuged at a speed within a range of 4000 rpm to 6000 rpm during a time period of 5 to 10 seconds.

8. An assay method for antimicrobial activity of an antimicrobial agent, comprising:
providing the antimicrobial agent to a culture medium in which strains are provided;
shaking the culture medium based on a first time interval, and sampling the culture medium based on a second time interval;
after the sampling of the culture medium, controlling movement of the culture medium such that the antimicrobial agent is to separate from a bacterial solution; and
after the separation of the antimicrobial agent from the bacterial solution, culturing the bacterial solution, and then determining information regarding the antimicrobial agent.

9. The assay method of claim 8, wherein the determined information includes a total number of microorganisms.

10. The assay method of claim 8, wherein a total amount of the antimicrobial agent provided to the culture medium is within a range of 0.02 g to 2 g.

11. The assay method of claim 8, wherein the strains are one selected from a group consisting of Klebsiella pneumoniae, Staphylococcus aureus, Micrococcus, Slalmonella typhimurium, Escherichia coli, Bacillus subtillus and Pseudomonas aeruginosa.

12. The assay method of claim 8, wherein the sampling of the culture medium includes sampling of the culture medium based on the second time interval such that a change in a total number of microorganisms is schematized temporally.

13. The assay method of claim 8, wherein the sampling of the culture medium is performed at 20 minute time intervals.

14. The assay method of claim 1, wherein the controlled movement of the culture medium is a speed within a range of 4000 rpm to 6000 rpm during a time period of 5 to 10 seconds.

15. An assay method for antimicrobial activity of an antimicrobial agent, comprising:
providing the antimicrobial agent at a culture medium in which strains are grown;
sampling, based on time, the culture medium having the provided antimicrobial agent;
after the sampling of the culture medium, separating the antimicrobial agent from a bacteria at the culture medium; and
after the separating of the antimicrobial agent from the bacteria, culturing the bacterial, and then determining information regarding the antimicrobial activity of the antimicrobial agent.

16. The assay method of claim 15, wherein the determining of the information regarding the antimicrobial activity of the antimicrobial agent includes determining a total number of microorganisms.

17. The assay method of claim 15, wherein the providing of the antimicrobial agent includes injecting a total amount of 0.02 g to 2 g of the antimicrobial agent into the culture medium.

18. The assay method of claim 15, wherein the sampling of the culture medium includes sampling the culture medium based on time such that a change in a total number of microorganisms is schematized temporally.

19. The assay method of claim 15, wherein the sampling is performed at time intervals.

20. The assay method of claim 15, wherein the separating of the antimicrobial agent from the bacteria at the culture medium includes centrifuging the culture medium at a speed within a range of 4000 to 6000 rpm during a time period of 5 to 10 seconds.
